(19) United States
(12) Patent Application Publication
(10) Pub. No.: US 2019/0066844 A1
(43) Pub. Date: Feb. 28, 2019

(54) FRACTURE INDEX DETERMINATION SYSTEM AND METHOD AND STORAGE MEDIUM

(71) Applicant: BOE Technology Group Co., Ltd., Beijing (CN)

(72) Inventors: Hailan Jin, Beijing (CN); Hui Du, Beijing (CN); Qi Yang, Beijing (CN); Yang Han, Beijing (CN)

(73) Assignee: BOE Technology Group Co., Ltd.

(21) Appl. No.: 16/018,415

(22) Filed: Jun. 26, 2018

(30) Foreign Application Priority Data
Aug. 24, 2017 (CN) 201710735552.0

(51) Int. Cl. G16H 50/30; G06T 7/20; A61B 5/00; G16H 20/30

(52) U.S. Cl. CPC G16H 50/30; G06T 7/20; G16H 20/30; A61B 5/4509; A61B 5/4023

(57) ABSTRACT

A fracture index determination system, method and storage medium are provided. The system includes: a bone mineral density (BMD) acquisition module, a balance degree acquisition module and a processing module. The BMD acquisition module and the balance degree acquisition module are connected to the processing module respectively; the BMD acquisition module is configured to acquire a BMD of a human body under test; the balance degree acquisition module is configured to acquire a balance degree of the human body under test, and the balance degree is used to characterize the standing stability of the human body; and the processing module is configured to determine a fracture index of the human body under test in accordance with the BMD and the balance degree of the human body under test. The system improves the accuracy of determining the fracture index.

This application claims priority to Chinese Patent Application No. 201710735552.0, filed with the State Intellectual Property Office on Aug. 24, 2017 and titled “Fracture Index Determination Method and System,” the entire contents of which are incorporated herein by reference.

### TECHNICAL FIELD

The present disclosure relates to a fracture index determination system, method and storage medium.

### BACKGROUND

With the improvement of living standards, people are paying more and more attention to bone health. Therefore, studies on bone health detection are indispensable. In these studies, it is often necessary to determine a fracture index, which is a parameter for measuring the likelihood of a bone fracture.

In the related art, a bone strength diagnostic device is usually adopted to determine the bone strength. Then, a medical specialist determines the fracture index in accordance with the bone strength. In particular, the bone strength diagnostic device comprises a sound velocity meter, an index calculator and a bone diagnostor. The index calculator is connected with the sound velocity meter and the bone diagnostor. The sound velocity meter is configured to radiate ultrasonic waves to bones and to measure the propagation velocity of the ultrasonic waves in the bones. The index calculator is configured to calculate the bone mineral density (BMD) in accordance with the propagation velocity of the ultrasonic waves in the bones. The bone diagnostor is configured to determine the bone strength in accordance with the BMD.

In the related art, the fracture index is determined by the medical specialist in accordance with the bone strength, and consequently, is impacted by human factors, which results in a relatively lower accuracy of the determined fracture index.

### SUMMARY

The present disclosure provides a fracture index determination system, method and storage medium.

In a first aspect, there is provided a fracture index determination system, comprising: a bone mineral density (BMD) acquisition module, a balance degree acquisition module and a processing module, wherein the BMD acquisition module and the balance degree acquisition module are connected to the processing module respectively; the BMD acquisition module is configured to acquire a BMD of a human body under test; the balance degree acquisition module is configured to acquire a balance degree of the human body under test, and the balance degree is used to characterize the standing stability of the human body; and the processing module is configured to determine a fracture index of the human body under test in accordance with the BMD and the balance degree of the human body under test.

Optionally, the fracture index determination system further comprises: a physical sign parameter acquisition module connected to the processing module and configured to acquire a physical sign parameter of the human body under test, wherein the processing module is configured to determine the fracture index of the human body under test in accordance with the BMD, the balance degree and the physical sign parameter of the human body under test.

Optionally, the balance degree comprises at least one of a static balance degree and a motion balance degree; the balance degree acquisition module comprises a bearing unit, a static balance degree acquisition unit and a motion balance degree acquisition unit, and the static balance degree acquisition unit and the motion balance degree acquisition unit are connected to the processing module respectively; the bearing unit is configured to bear the human body under test; the static balance degree acquisition unit is configured to acquire a static balance degree of the human body under test when the human body under test is in a static state on the bearing unit; and the motion balance degree acquisition unit is configured to acquire a motion balance degree of the human body under test when the human body under test is in a moving state on the bearing unit.

Optionally, the static balance degree acquisition unit comprises a pressure sensor and a processing sub-unit, and the pressure sensor is disposed on the bearing unit and connected to the processing sub-unit; the pressure sensor is configured to acquire pressure applied by the human body under test to the bearing unit when the human body under test is in the static state on the bearing unit; and the processing sub-unit is configured to determine a variation of the pressure applied by the human body under test to the bearing unit in accordance with the pressure applied by the human body under test to the bearing unit within a test period and to determine the static balance degree of the human body under test in accordance with the variation of the pressure.

Optionally, the motion balance degree acquisition unit comprises a camera and a processing sub-unit, and the camera is disposed in a target position, a preset distance away from the bearing unit, and is connected to the processing sub-unit; the camera is configured to acquire a motion image of the human body under test when the human body under test is in the moving state on the bearing unit; and the processing sub-unit is configured to determine motion information of the human body under test in accordance with the motion image of the human body under test and to determine the motion balance degree of the human body under test in accordance with the motion information of the human body under test.

Optionally, the fracture index determination system further comprises a motion item determination module connected to the motion balance degree acquisition unit; the motion item determination module is configured to determine a motion item that corresponds to the human body under test in accordance with the BMD or the physical sign parameter of the human body under test; and the motion balance degree acquisition unit is configured to acquire the motion balance degree of the human body under test when the human body under test performs the motion item that corresponds to the human body under test on the bearing unit.

Optionally, the fracture index determination system further comprises a three-dimensional motion capture module and a presentation module that are connected to the processing module respectively; the three-dimensional motion capture module is configured to capture a motion action of the human body under test when the human body under test performs the motion item that corresponds to the human body under test on the bearing unit; the processing module is configured to generate test guidance information in accordance with the motion action of the human body under test, and the test guidance information is used to guide the human body under test to finish the motion item that corresponds to the human body under test; and the presentation module is configured to present the test guidance information.

Optionally, the processing module is further configured to determine an exercise plan for the human body under test in accordance with the fracture index of the human body under test; and the presentation module is further configured to present the exercise plan.

Optionally, the processing module is configured to determine the fracture index of the human body under test in accordance with the BMD, the balance degree of the human body under test and a fracture index model.

Optionally, the fracture index determination system further comprises a communication module connected to the processing module, wherein the communication module is configured to upload the fracture index of the human body under test and the exercise plan for the human body under test to a server.

In another aspect, there is provided a fracture index determination method, applied to the fracture index determination systems described above or any of the fracture index determination systems described above. The method comprises: acquiring a bone mineral density (BMD) of a human body under test; acquiring a balance degree of the human body under test, wherein the balance degree is used to characterize the standing stability of the human body; and determining a fracture index of the human body under test in accordance with the BMD and the balance degree of the human body under test.

Optionally, the method further comprises: acquiring a physical sign parameter of the human body under test, wherein said determining the fracture index of the human body under test in accordance with the BMD and the balance degree of the human body under test comprises: determining the fracture index of the human body under test in accordance with the BMD, the balance degree and the physical sign parameter of the human body under test.

Optionally, said acquiring the balance degree of the human body under test comprises: acquiring a static balance degree of the human body under test when the human body under test is in a static state on a bearing unit; and acquiring a motion balance degree of the human body under test when the human body under test is in a moving state on the bearing unit.

Optionally, said acquiring the static balance degree of the human body under test when the human body under test is in the static state on the bearing unit comprises: acquiring pressure applied by the human body under test to the bearing unit when the human body under test is in the static state on the bearing unit; and determining a variation of the pressure applied by the human body under test to the bearing unit in accordance with the pressure applied by the human body under test to the bearing unit within a test period and determining the static balance degree of the human body under test in accordance with the variation of the pressure.

Optionally, said acquiring the motion balance degree of the human body under test when the human body under test is in the moving state on the bearing unit comprises: acquiring a motion image of the human body under test when the human body under test is in the moving state on the bearing unit; and determining motion information of the human body under test in accordance with the motion image of the human body under test and determining the motion balance degree of the human body under test in accordance with the motion information of the human body under test.

Optionally, the method further comprises: determining a motion item that corresponds to the human body under test in accordance with the BMD or the physical sign parameter of the human body under test, wherein said acquiring the motion balance degree of the human body under test when the human body under test is in the moving state on the bearing unit comprises: acquiring the motion balance degree of the human body under test when the human body under test performs the motion item that corresponds to the human body under test on the bearing unit.

Optionally, the method further comprises: capturing a motion action of the human body under test when the human body under test performs the motion item that corresponds to the human body under test on the bearing unit generating test guidance information in accordance with the motion action of the human body under test, wherein the test guidance information is used to guide the human body under test to perform the motion item that corresponds to the human body under test; and presenting the test guidance information.

Optionally, the method further comprises: determining an exercise plan for the human body under test in accordance with the fracture index of the human body under test; and presenting the exercise plan.

Optionally, said determining the fracture index of the human body under test in accordance with the BMD and the balance degree of the human body under test comprises: determining the fracture index of the human body under test by means of a processing module in accordance with the BMD, the balance degree of the human body under test and a fracture index model.

In yet another aspect, there is provided a non-temporary computer readable storage medium comprising instructions therein. When the instruction in the non-temporary computer readable storage medium is operated on a processing component, the processing component executes the following steps: acquiring a bone mineral density (BMD) of a human body under test; acquiring a balance degree of the human body under test, wherein the balance degree is used to characterize the standing stability of the human body; and determining a fracture index of the human body under test in accordance with the BMD and the balance degree of the human body under test.

With the fracture index determination system, method and storage medium provided by the embodiments of the present disclosure, the BMD acquisition module may acquire the BMD of the human body, the balance degree acquisition module may acquire the balance degree of the human body, and the processing module may determine the fracture index of the human body in accordance with the BMD and the balance degree of the human body.

### BRIEF DESCRIPTION OF THE DRAWINGS

FIG. 1 is a block diagram of a fracture index determination system provided in an embodiment of the present disclosure;

FIG. 2 is a schematic diagram of a partial structure of a fracture index determination system provided in an embodiment of the present disclosure;

FIG. 3 is a state diagram of a partial structure of a fracture index determination system provided in an embodiment of the present disclosure;

FIG. 4 is a state diagram of a partial structure of another fracture index determination system provided in an embodiment of the present disclosure;

FIG. 5 is a block diagram of another fracture index determination system provided in an embodiment of the present disclosure; and

FIG. 6 is a flowchart of a fracture index determination method provided in an embodiment of the present disclosure.

### DETAILED DESCRIPTION

The present disclosure will be described in further detail with reference to the enclosed drawings, to clearly present the principles and advantages of the present disclosure. Apparently, the embodiments described are only some embodiments of the present disclosure, rather than all embodiments of the present disclosure.

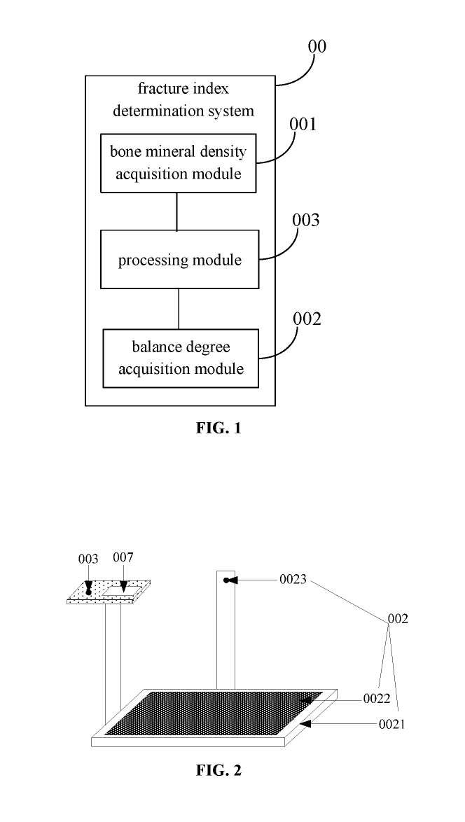

Please refer to FIG. 1, which shows a block diagram of a fracture index determination system 00 provided in an embodiment of the present disclosure. Referring to FIG. 1, the fracture index determination system 00 may include: a bone mineral density (BMD) acquisition module 001, a balance degree acquisition module 002 and a processing module 003, wherein the BMD acquisition module 001 and the balance degree acquisition module 002 are connected to the processing module 003 respectively.

The bone mineral density (BMD) acquisition module 001 is configured to acquire a BMD of a human body under test. The balance degree acquisition module 002 is configured to acquire a balance degree of the human body under test. The balance degree is used to characterize the standing stability of the human body. The processing module 003 is configured to determine a fracture index of the human body under test in accordance with the BMD and the balance degree of the human body under test. The fracture index may be configured to indicate the risk of a bone fracture of the human body under test. The higher the fracture index is, the higher the risk of the bone fracture is.

To sum up, in the fracture index determination system provided by the embodiments of the present disclosure, the BMD acquisition module may acquire the BMD of the human body under test. The balance degree acquisition module may acquire the balance degree of the human body under test. The processing module may determine the fracture index of the human body under test in accordance with the BMD and the balance degree of the human body under test. In the embodiments of the present disclosure, as the fracture index is determined through the BMD and the balance degree of the human body under test, the problem of a relatively lower accuracy in determining the fracture index is solved. The accuracy in determining the fracture index is improved.

Optionally, in the embodiments of the present disclosure, the fracture index may be in negative correlation with the BMD and may also be in negative correlation with the balance degree. That is, the lower the BMD is and the lower the balance degree is, the higher the fracture index is, and the higher the fracture probability of the human body under test is.

Optionally, the BMD acquisition module 001 may acquire the BMD of the human body under test based on any of the quantitative ultrasound technology, the one-photon absorption technology, the two-photon absorption technology, the quantitative computed tomography (CT) technology and the dual-energy X-ray absorptiometry technology. Exemplarily, the BMD acquisition module 001 may acquire the BMD of the human body under test based on the dual-energy X-ray absorptiometry technology. For example, the BMD acquisition module 001 may comprise an X-ray source, a detector and a processor. The X-ray source may emit X-rays to a bone of the human body under test. The detector may receive the X-rays penetrating the bone of the human body under test and may send the received amount of rays to the processor. The processor may determine the amount of rays absorbed by the bone of the human body under test in accordance with the amount of rays emitted by the X-ray source and the amount of rays received by the detector and may further determine the BMD in accordance with the received amount of rays.

Optionally, in the embodiments of the present disclosure, the balance degree may comprise at least one of a static balance degree and a motion balance degree. The static balance degree is a balance degree that is measured when the human body under test is in a static state, and for example, may be a balance degree that is measured when feet of the human body under test are in a static state. The motion balance degree is a balance degree that is measured when the human body under test is in a moving state, and for example, may be a balance degree that is measured when the feet of the human body under test are in a moving state.

Referring to FIG. 2, which is a partial schematic structural view of a fracture index determination system 00 provided by an embodiment of the present disclosure, the balance degree acquisition module 002 comprises a bearing unit 0021, a static balance degree acquisition unit (not shown in FIG. 2) and a motion balance degree acquisition module (not shown in FIG. 2). The static balance degree acquisition unit and the motion balance degree acquisition unit are connected to the processing module (not shown in FIG. 2).

The bearing unit 0021 is configured to bear the human body under test. The static balance degree acquisition unit is configured to acquire a static balance degree of the human body under test when the human body under test is in a static state on the bearing unit 0021. The motion balance degree acquisition unit is configured to acquire a motion balance degree of the human body under test when the human body under test is in a moving state on the bearing unit 0021. The bearing unit 0021 may be a bearing board. An angle between a bearing face of the bearing unit 0021 and a horizontal plane may be adjusted. That is, the slope (also called inclination) of the bearing unit 0021 may be adjusted. Exemplarily, a groove is formed in a back side (a side, opposite to the bearing face, of the bearing unit 0021) of the bearing unit 0021. A lifting column is disposed in the groove and fixedly secured to an edge of the back side of the bearing unit 0021. The lifting column may be controlled to ascend and descend so as to enable one end of the bearing unit 0021 to rise and fall. Thus, a certain angle is formed between the bearing face of the bearing unit 0021 and the horizontal plane. Optionally, the lifting column may be a pneumatic full-automatic lifting column.

The static balance degree acquisition unit may perform a static balance test on the human body under test to acquire the static balance degree. As shown in FIG. 2, the static balance degree acquisition unit comprises a pressure sensor 0022 and a processing sub-unit (not shown in FIG. 2). The pressure sensor 0022 is disposed on the bearing unit 0021 and connected to the processing sub-unit (not shown in FIG. 2). For example, the pressure sensor 0022 may be a sensor array and is configured to acquire pressure applied by the human body under test to the bearing unit when the human body under test is in the static state on the bearing unit 0021. The processing sub-unit is configured to determine a variation of the pressure applied by the human body under test to the bearing unit 0021 in accordance with the pressure applied by the human body under test to the bearing unit 0021 within a test period, and to determine the static balance degree of the human body under test in accordance with the variation of the pressure.

Optionally, when the static balance test is required, the test time may be set in the processing sub-unit in advance, and may be a preset time (for example, 5 min). When standing on the bearing unit 0021, the human body under test may apply pressure to the bearing unit 0021. The pressure sensor 0022 may continuously acquire the pressure applied by the human body under test to the bearing unit 0021 and send the acquired pressure to the processing sub-unit. The processing sub-unit may determine variation (for example, a pressure graph) of the pressure applied by the human body under test to the bearing unit 0021 in accordance with the pressure sent by the pressure sensor 0022 within the preset time (for example, 5 min), and may also determine the center of the pressure applied by the human body under test to the bearing unit 0021 in accordance with the variation of the pressure. The center of pressure (COP) may also be called a ground reaction force center. Furthermore, the processing sub-unit may determine the static balance degree of the human body under test in accordance with offset of the center of pressure to center of mass of the human body under test.

Optionally, the static balance degree and the offset may be in negative correlation. That is, the higher the offset is, the lower the static balance degree is, indicating a poorer stability of the human body under test.

In the embodiments of the present disclosure, the static balance test may comprise at least one of a single-foot standing test and a functional reaching test. When the single-foot standing test is required, the human body under test stands on one foot on the bearing unit 0021 and is physically relaxed naturally. The pressure sensor 0022 continuously acquires pressure applied by the human body under test to the bearing unit 0021 and sends the acquired pressure to the processing sub-unit. The processing sub-unit determines the static balance degree of the human body in accordance with the pressure sent by the pressure sensor 0022. When the functional reaching test is required, the human body under test stands on both feet on the bearing unit 0021 and extends arms forwards. The pressure sensor 0022 continuously acquires the pressure applied by the human body under test to the bearing unit 0021 and sends the acquired pressure to the processing sub-unit. The processing sub-unit determines the static balance degree of the human body in accordance with the pressure sent by the pressure sensor 0022.

The motion balance degree acquisition unit may perform a motion balance test on the human body under test to acquire the motion balance degree. As shown in FIG. 2, the motion balance degree acquisition unit comprises a camera 0023 and a processing sub-unit (not shown in FIG. 2). The camera 0023 is disposed in a target position, a preset distance away from the bearing unit 0021, and is connected to the processing sub-unit (not shown in FIG. 2). The camera 0023 is configured to acquire a motion image of the human body under test when the human body under test is in a moving state on the bearing unit 0021, and to send the acquired motion image of the human body under test to the processing sub-unit. The processing sub-unit is configured to determine motion information of the human body under test in accordance with the motion image of the human body under test, and to determine the motion balance degree of the human body under test in accordance with the motion information of the human body under test.

Optionally, when the motion balance test is required, a test period may be set in the processing sub-unit in advance, and may be a preset time (for example, 10 min). When the human body under test moves on the bearing unit 0021, the camera 0023 may continuously acquire the motion image of the human body under test and send the acquired motion image to the processing sub-unit. The processing sub-unit analyzes the motion image sent by the camera 0023 within the preset time (for example, 10 min) to obtain the motion information of the human body under test. The motion information may comprise at least one of step speed, step size and percentage of single-foot support time. A corresponding relationship between the motion information and the motion balance degree may be stored in the processing sub-unit. The corresponding relationship between the motion information and the motion balance degree is inquired in accordance with the motion information obtained through analysis to obtain the motion balance degree of the human body under test.

The corresponding relationship between the motion information and the motion balance degree may be set in the processing sub-unit in advance by a medical specialist and may also be obtained in the way that the processing sub-unit performs balance degree training on sample data in advance. In the embodiments of the present disclosure, the step speed may be the average step speed at which the human body under test moves on the bearing unit 0021. The step size may be the average step size at which the human body under test moves on the bearing unit 0021. The percentage of single-foot support time may be the percentage of single-foot ground contact time to the total motion time when the human body under test moves on the bearing unit 0021.

In the embodiments of the present disclosure, the motion balance test may comprise at least one of a platform motion balance degree test, a slope motion balance degree test and an across-obstacle motion balance degree test. When the platform motion balance degree test is required, as shown in FIG. 2, the bearing face of the bearing unit 0021 is kept parallel to the horizontal plane. The human body under test (not shown in FIG. 2) stands on the bearing unit 0021 and moves (for instance, walks slowly, walks fast and runs). The camera 0023 acquires the motion image of the human body under test and sends the acquired motion image to the processing sub-unit. The processing sub-unit analyzes the motion image sent by the camera 0023 to obtain the motion information of the human body under test, and inquires the corresponding relationship between the motion information and the motion balance degree to obtain the motion balance degree of the human body under test.

When the slope motion balance degree test is required, as shown in FIG. 3, the height of one edge of the bearing unit 0021 is adjusted to form an angle α (the bearing face of the bearing unit 0021 has a certain slope) between the bearing unit 0021 and the horizontal plane. The human body under test (not shown in FIG. 3) stands on the bearing unit 0021 and moves (for instance, walks slowly, walks fast and runs, etc.). The camera 0023 acquires the motion image of the human body under test and sends the acquired motion image to the processing sub-unit. The processing sub-unit analyzes the motion image sent by the camera 0023 to obtain the motion information of the human body under test, and inquires the corresponding relationship between the motion information and the motion balance degree to obtain the motion balance degree of the human body under test.

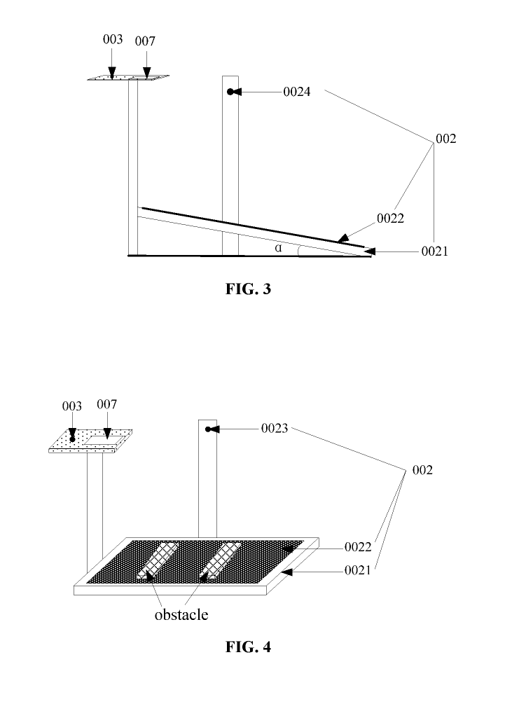

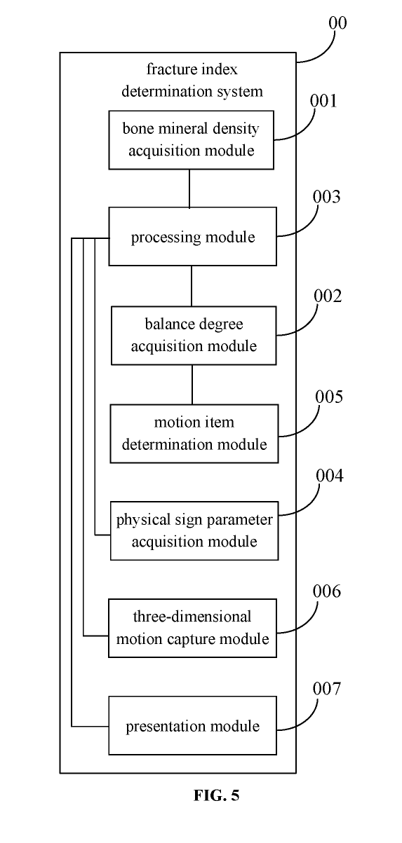

When the across-obstacle motion balance degree test is required, as shown in FIG. 4, the bearing face of the bearing unit 0021 is kept parallel to the horizontal plane (or, a certain angle is formed between the bearing face of the bearing unit 0021 and the horizontal plane). An obstacle is disposed on the bearing face of the bearing unit 0021. The human body under test (not shown in FIG. 4) stands on the bearing unit 0021 and moves across the obstacle (for instance, walks slowly, walks fast or runs, etc.). The camera 0023 acquires the motion image of the human body under test and sends the acquired motion image to the processing sub-unit. The processing sub-unit analyzes the motion image sent by the camera 0023 to obtain the motion information of the human body under test, and inquires the corresponding relationship between the motion information and the motion balance degree to obtain the motion balance degree of the human body under test.

Furthermore, referring to FIG. 5, which shows a block diagram of another fracture index determination system 00 provided by an embodiment of the present disclosure. Referring to FIG. 5, on the basis of the structure as shown in FIG. 1, the fracture index determination system 00 further comprises a physical sign parameter acquisition module 004 that is connected to a processing module 003.

The physical sign parameter acquisition module 004 is configured to acquire a physical sign parameter of a human body under test and sends the acquired physical sign parameter to the processing module 003. The processing module 003 is configured to determine a fracture index of the human body under test in accordance with a bone mineral density (BMD), a balance degree and the physical sign parameter of the human body under test.

The physical sign parameter may comprise but not limited to at least one of age, height, weight and a health parameter. The health parameter is used to indicate a health condition of the human body under test. The health condition may mean whether the human body under test suffers from a disease and what kind of disease (for instance, hypertension and heart disease, etc.) the human body under test suffers from. Besides, the health parameter may adopt different data to indicate whether the human body under test suffers from a disease and what kind of disease the human body under test suffers from. Optionally, the physical sign parameter may further comprise parameters including body temperature, pulse, breathe, blood pressure and the like. The types of the parameters included by the physical sign parameter will not be limited by the embodiments of the present disclosure.

In the embodiments of the present disclosure, the physical sign parameter acquisition module 004 may be an input module. A user may input the physical sign parameter of the human body under test by means of the input module, so that the physical sign parameter acquisition module 004 may acquire the physical sign parameter. Optionally, the user may input parameters including sex and the like of the human body under test by means of the input module, which will not be limited by the embodiments of the present disclosure.

The processing module 003 may determine the fracture index of the human body under test in accordance with the BMD, the balance degree and the physical sign parameter of the human body under test. Optionally, the processing module 003 may determine the fracture index of the human body under test in accordance with the BMD, the balance degree of the human body under test and a fracture index model. The fracture index model may be built and provided in the processing module 003 in advance. The fracture index model may be built in the way that physical sign parameters, BMDs, balance degrees and fracture indexes of m human bodies are acquired to obtain m groups of data. Each of the m groups of data comprise the physical sign parameter, the BMD, the balance degree and the fracture index and correspond to one human body. The m groups of data are analyzed to obtain the fracture index model. Exemplarily, a linear equation may be built in accordance with each group of data. Thus, m linear equations are obtained and may comprise:

$$\{ \begin{matrix}{f}_{1} = {k}_{1} \times {a}_{1} + {k}_{2} \times {h}_{1} + {k}_{3} \times {g}_{1} + {k}_{4} \times {d}_{1} + {k}_{5} \times {b}_{1} + {k}_{6} \times {j}_{1} + {k}_{7} \\ {f}_{2} = {k}_{1} \times {a}_{2} + {k}_{2} \times {h}_{2} + {k}_{3} \times {g}_{2} + {k}_{4} \times {d}_{2} + {k}_{5} \times {b}_{2} + {k}_{6} \times {j}_{2} + {k}_{7} \\ … \\ {f}_{m} = {k}_{1} \times {a}_{m} + {k}_{2} \times {h}_{m} + {k}_{3} \times {g}_{m} + {k}_{4} \times {d}_{m} + {k}_{5} \times {b}_{m} + {k}_{6} \times {j}_{m} + {k}_{7}\end{matrix} ,$$

where a₁ represents the age of a human body 1 in the m human bodies, h₁ represents the height of the human body 1 in the m human bodies, g₁ represents the weight of the human body 1 in the m human bodies, d₁ represents the BMD of the human body 1 in the m human bodies, b₁ represents the balance degree of the human body 1 in the m human bodies, j₁ represents the health parameter of the human body 1 in the m human bodies, and f₁ represents the fracture index of the human body 1. Similarly, a₂ represents the age of a human body 2 in the m human bodies, h₂ represents the height of the human body 2 in the m human bodies, g₂ represents the weight of the human body 2 in the m human bodies, d₂ represents the BMD of the human body 2 in the m human bodies, b₂ represents the balance degree of the human body 2 in the m human bodies, j₂ represents the health parameter of the human body 2 in the m human bodies, f₂ represents the fracture index of the human body 2, and so on. k1, k2, k3, k4, k5 and k6 are sequentially coefficients of age, height, weight, BMD, balance degree and health parameter. k7 is an additional coefficient.

The m linear equations are solved to obtain specific values of k1, k2, k3, k4, k5, k6 and k7. Then, the fracture index model may be obtained in accordance with the values of k1, k2, k3, k4, k5, k6 and k7, and may be: f=k₁×a+k₂×h+k₃×g+k₄×d+k₅×b+k₆×j+k₇, where a, h, g, d, b and j are all unknown numbers and sequentially represent age, height, weight, BMD, balance degree and health parameter. After the fracture index model is built, the processing module may substitute the BMD, the balance degree and the physical sign parameter of the human body under test into the fracture index model. The fracture index of the human body under test is obtained through calculation.

It should be noted that the above-mentioned solution of building the fracture index model is merely exemplary. In the embodiments of the present disclosure, the fracture index model may be built by other means. For example, the fracture index model may be built by a support vector machine (SVM) algorithm, a neural network algorithm or the like. The fracture index model may also be a back propagation (BP) neutral network model.

In the embodiments of the present disclosure, the value range of the fracture index may be [0,10]. 0 indicates no fracture risk of the human body. 10 indicates an extremely high fracture risk of the human body.

Furthermore, continuously referring to FIG. 5, the fracture index determination system 00 may further comprise a motion item determination module 005 that is connected to the motion balance degree acquisition unit (not shown in FIG. 5). The motion item determination module is configured to determine the motion item that corresponds to the human body under test in accordance with the BMD or the physical sign parameter of the human body under test. The motion balance degree acquisition unit is configured to acquire the motion balance degree of the human body under test when the human body under test performs the motion item that corresponds to the human body under test on the bearing unit.

The motion item may comprise motion type and motion intensity. The motion type is test type that may comprise the platform motion balance degree test, the slope motion balance degree test and the across-obstacle motion balance degree test. The motion intensity may comprise primary intensity, secondary intensity, tertiary intensity and the like. The primary intensity indicates a relatively lower motion intensity (for example, slow walking). The secondary intensity indicates an appropriate motion intensity (for example, fast walking). The tertiary intensity indicates a relatively higher motion intensity (for example, running).

Optionally, a BMD threshold may be set in the motion item determination module 005 which may determine the motion item that corresponds to the human body under test in accordance with the relationship between the BMD of the human body under test and the BMD threshold. Optionally, when the BMD of the human body under test is more than or equal to the BMD threshold, it is believed that the human body under test has a relatively higher BMD. At this time, the motion item determination module 005 determines the motion item with the relatively higher motion intensity as the motion item that corresponds to the human body under test. For example, the slope motion balance degree test item with the tertiary motion intensity is determined as the motion item that corresponds to the human body under test. When the BMD of the human body under test is less than the BMD threshold, it is believed that the human body under test has a relatively lower BMD. At this time, the motion item determination module 005 determines the motion item with the relatively lower motion intensity as the motion item that corresponds to the human body under test. For example, the platform motion balance degree test item with the primary motion intensity is determined as the motion item that corresponds to the human body under test.

Optionally, a corresponding relationship between the physical sign parameter and the motion item may be stored in the motion item determination module 005, which may inquire the corresponding relationship between the physical sign parameter and the motion item in accordance with the physical sign parameter of the human body under test to obtain the motion item that corresponds to the human body under test. Exemplarily, when the physical sign parameter is the health parameter, the motion item with the relatively lower motion intensity may be determined as the motion item that corresponds to the human body under test if the health parameter indicates that the human body under test suffers from hypertension or heart disease. For example, the platform motion balance degree test item with the primary motion intensity is determined as the motion item that corresponds to the human body under test.

Furthermore, continuously referring to FIG. 5, the fracture index determination system 00 may further comprise a three-dimensional motion capture module 006 and a presentation module 007 that are connected to the processing module 003.

The three-dimensional motion capture module 006 is configured to capture a motion action of the human body under test when the human body under test performs the motion item that corresponds to the human body under test on the bearing unit. The processing module 003 is configured to generate test guidance information in accordance with the motion action of the human body under test. The test guidance information is used to guide the human body under test to finish the motion item that corresponds to the human body under test. The presentation module 007 is configured to present the test guidance information.

The three-dimensional motion capture module 006 may comprise a plurality of cameras. The cameras may continuously capture the motion action of the human body under test when the human body under test performs the motion item that corresponds to the human body under test on the bearing unit. Action information for indicating the motion action is generated in accordance with the motion action of the human body under test and sent to the processing module 003. The processing module 003 may compare the action information sent by the three-dimensional motion capture module 006 with self-stored action information to determine whether there exists a motion action that is identical with the motion action of the human body under test in the motion action indicated by the self-stored action information or not. If yes, the processing module 003 determines the motion action of the human body under test as a correct motion action. If not, the processing module 003 determines the motion action of the human body under test as a false motion action. At this time, the processing module 003 may generate the test guidance information that is configured to indicate the motion action. The motion action indicated by the test guidance information is the motion action that is nearest to the motion action of the human body under test in the motion action indicated by the action information stored in the processing module 003.

For example, the presentation module 007 may be a display module or a voice broadcasting module. The display module may be an organic light-emitting diode (OLED) display device or a liquid crystal display (LCD). The presentation module 007 may present the test guidance information in the format of text displaying, image displaying, voice broadcasting or video displaying to guide the human body under test to make the correct motion action. For example, the presentation module 007 may present a video that includes the correct motion action.

In the embodiments of the present disclosure, the processing module 003 is further configured to determine an exercise plan for the human body under test in accordance with the fracture index of the human body under test. The presentation module 007 may be further configured to present the exercise plan.

Optionally, a corresponding relationship between a fracture index interval and the exercise plan may be stored in the processing module 003. The processing module 003 may determine the fracture index interval to which the fracture index of the human body under test belongs first, and then inquires the corresponding relationship between the fracture index interval and the exercise plan in accordance with the fracture index interval to which the fracture index of the human body under test belongs to obtain the exercise plan for the human body under test.

Exemplarily, in the embodiments of the present disclosure, the value range of the fracture index may be [0, 10] and may comprise a plurality of fracture index intervals that may comprise (0, 1], (1, 2], (2, 3], (3, 4] and the like. An exercise plan that corresponds to each of the plurality of fracture index intervals may be stored in the processing module 003. Supposing that the fracture index of the human body under test belongs to the fracture index interval (8, 9], the processing module 003 may determine an exercise plan that corresponds to (8, 9] as the exercise plan for the human body under test. The exercise plan may comprise a life advice. Exemplarily, the exercise plan for the human body under test may be that calcium supplement is recommended when the human body under test has a relatively higher fracture risk. In some embodiments, the exercise plan for the human body under test may be that an intensive balance training is recommended when the human body under test is likely to fall over. In some embodiments, the exercise plan for the human body under test may be that the human body under test may not climb stairs independently or lift weights.

Optionally, the corresponding relationship between the fracture index interval and the exercise plan may be preset in the processing module 003 by a medical specialist and may also be obtained in the way that the processing module 003 performs training on sample data, which will not be limited by the embodiments of the present disclosure.

The presentation module 007 is configured to present the exercise plan after the processing module 003 determines the exercise plan for the human body under test. Optionally, the presentation module 007 may present the exercise plan in the format of text displaying or voice broadcasting. Exemplarily, the presentation module 007 may be a display screen, a voice player or the like.

Optionally, in the embodiments of the present disclosure, as shown in FIGS. 2 to 4, the balance degree acquisition module, the processing module 003 and the presentation module 007 may be integrated or disposed independently, which will not be limited by the embodiments of the present disclosure.

Optionally, the fracture index determination system provided by the embodiments of the present disclosure may also upload the fracture index and the exercise plan of the human body under test to a server. For example, the fracture index determination system may further comprise a communication module that is connected to the processing module. The communication module may upload the fracture index and the exercise plan that are determined by the processing module to the server which may store the fracture index and the exercise plan of the human body under test. In the following, the fracture index determination system may acquire the fracture index and the exercise plan of the human body under test from the server by means of the communication module. Thus, a storage resource of the fracture index determination system may not be wasted.

To sum up, in the fracture index determination system provided by the embodiments of the present disclosure, the BMD acquisition module may acquire the BMD of the human body under test. The balance degree acquisition module may acquire the balance degree of the human body under test. The processing module may determine the fracture index of the human body under test in accordance with the BMD and the balance degree of the human body under test. In the embodiments of the present disclosure, as the fracture index is determined through the BMD and the balance degree of the human body under test, the problem of a relatively lower accuracy in determining the fracture index is solved. The accuracy in determining the fracture index is improved.

The fracture index determination system provided by the embodiments of the present disclosure may be applied to such scenarios as monitoring of pregnant women, health protection for the aged and rehabilitation of people injured in sports. Optionally, the fracture index determination system may be applied to a physical examination center or a health screening mechanism and is configured to evaluate the fracture risk of the user and to give suggestions. Alternatively, the fracture index determination system may serve as a household appliance for long-time evaluation of the health risk of the user.

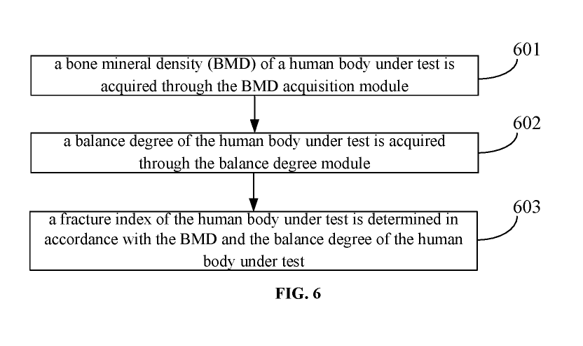

Please refer to FIG. 6, which shows a flowchart of a fracture index determination method provided in an embodiment of the present disclosure. The fracture index determination method may be applied to the fracture index determination system described above. For example, the method may be implemented by the processing module 003 in the fracture index determination system 00 shown in FIG. 1 or FIG. 5. The embodiment is illustrated by taking an example in which the fracture index determination method is applied to the processing module 003 in the fracture index determination system 00 shown in FIG. 5. Referring to FIG. 5, the fracture index determination system 00 further includes: a bone mineral density (BMD) acquisition module 001 and a balance degree acquisition module 002. The BMD acquisition module 001 and the balance degree acquisition module 002 are connected to the processing module 003 respectively. Referring to FIG. 6, the method may include the following steps.

In step 601, a bone mineral density (BMD) of a human body under test is acquired through the BMD acquisition module.

In step 602, a balance degree of the human body under test is acquired through the balance degree module.

In step 603, a fracture index of the human body under test is determined in accordance with the BMD and the balance degree of the human body under test.

To sum up, in the fracture index determination method provided by the embodiments of the present disclosure, the processing module may acquire the BMD of the human body under test through the BMD acquisition module, acquire the balance degree of the human body under test through the balance degree acquisition module and determine the fracture index of the human body under test in accordance with the BMD and the balance degree of the human body under test. In the embodiments of the present disclosure, as the fracture index is determined through the BMD and the balance degree of the human body under test, the problem of a relatively lower accuracy in determining the fracture index is solved. The accuracy in determining the fracture index is improved.

The BMD acquisition module may acquire the BMD of the human body under test based on any of the quantitative ultrasound technology, the one-photon absorption technology, the two-photon absorption technology, the quantitative computed tomography (CT) technology and the dual-energy X-ray absorptiometry technology. Exemplarily, the BMD acquisition module comprises an X-ray source, a detector and a processor. The X-ray source may emit X-rays to a bone of the human body under test. The detector may receive the X-rays that penetrate the bone of the human body under test and may send the received rays to the processor. The processor may determine the amount of rays absorbed by the bone of the human body under test in accordance with the amount of rays emitted by the X-ray source and the amount of rays received by the detector and may further determine the BMD in accordance with the received amount of rays.

Optionally, the balance degree may comprise at least one of a static balance degree and a motion balance degree. The static balance degree is a balance degree that is measured when the human body under test is in a static state, and for example, may be a balance degree that is measured when feet of the human body under test are in a static state. The motion balance degree is a balance degree that is measured when the human body under test is in a moving state, and for example, may be a balance degree that is measured when the feet of the human body under test are in a moving state.

The balance degree acquisition module comprises a bearing unit, a static balance degree acquisition unit and a motion balance degree acquisition unit. The static balance degree acquisition unit and the motion balance degree acquisition unit are connected to the processing module. The step 602 may comprise: bearing the human body under test by means of the bearing unit; acquiring a static balance degree of the human body under test by means of the static balance degree acquisition unit when the human body under test is in a static state on the bearing unit; and acquiring a motion balance degree of the human body under test by means of the motion balance degree acquisition unit when the human body under test is in a moving state on the bearing unit.

Optionally, the static balance degree acquisition unit comprises a pressure sensor and a processing sub-unit. The pressure sensor is disposed on the bearing unit and connected to the processing sub-unit.

The procedure of acquiring the static balance degree of the human body under test by means of the static balance degree acquisition unit when the human body under test is in the static state on the bearing unit may comprise: acquiring pressure applied by the human body under test to the bearing unit when the human body under test is in the static state on the bearing unit; and determining a variation of the pressure applied by the human body under test to the bearing unit in accordance with the pressure applied by the human body under test to the bearing unit within a test period and determining the static balance degree of the human body under test in accordance with the variation of the pressure.

Optionally, when a static balance degree test is required, the test time may be set in the processing sub-unit in advance. When standing on the bearing unit, the human body under test may apply pressure to the bearing unit. The pressure sensor may continuously acquire the pressure applied by the human body under test to the bearing unit and send the acquired pressure to the processing sub-unit. The processing sub-unit may determine variation of the pressure applied by the human body under test to the bearing unit in accordance with the pressure sent by the pressure sensor within the preset time, and may also determine the center of the pressure applied by the human body under test to the bearing unit in accordance with the variation of the pressure. The processing sub-unit may determine the static balance degree of the human body under test in accordance with offset of the center of pressure to center of mass of the human body under test.

Optionally, the motion balance degree acquisition unit comprises a camera and a processing sub-unit. The camera is disposed in a target position, a preset distance away from the bearing unit, and is connected to the processing sub-unit.

The procedure of acquiring the motion balance degree of the human body under test by means of the motion balance degree acquisition unit when the human body under test is in the moving state on the bearing unit may comprise: acquiring a motion image of the human body under test by means of the camera when the human body under test is in the moving state on the bearing unit; and determining motion information of the human body under test by means of the processing sub-unit in accordance with the motion image of the human body under test, and determining the motion balance degree of the human body under test in accordance with the motion information of the human body under test.

Optionally, when a motion balance test is required, a test period may be set in the processing sub-unit in advance. The camera may continuously acquire a motion image of the human body under test and send the acquired motion image to the processing sub-unit when the human body under test moves on the bearing unit. The processing sub-unit analyzes the motion image sent by the camera within a preset time to obtain motion information of the human body under test. The motion information may comprise at least one of step speed, step size and percentage of single-foot support time. A corresponding relationship between the motion information and the motion balance degree may be stored in the processing sub-unit, and is inquired in accordance with the motion information obtained through analysis to obtain the motion balance degree of the human body under test.

The corresponding relationship between the motion information and the motion balance degree may be set in the processing sub-unit in advance by a medical specialist and may also be obtained in the way that the processing sub-unit performs balance degree training on sample data in advance. In the embodiments of the present disclosure, the step speed may be the average step speed at which the human body under test moves on the bearing unit. The step size may be the average step size at which the human body under test moves on the bearing unit. The percentage of single-foot support time may be the percentage of single-foot ground contact time to the total motion time when the human body under test moves on the bearing unit.

Furthermore, continuously referring to FIG. 5, the fracture index determination system further comprises a physical sign parameter acquisition module 004 that is connected to a processing module 003. Before the step 603, the fracture index determination method further comprises: acquiring a physical sign parameter of the human body under test by means of the physical sign parameter acquisition module. The step 603 may comprise: determining a fracture index of the human body under test in accordance with the BMD, the balance degree and the physical sign parameter of the human body under test.

For example, the physical sign parameter may comprise but not limited to at least one of age, height, weight and a health parameter. The health parameter is used to indicate a health condition of the human body under test. The health condition may mean whether the human body under test suffers from a disease and what kind of disease the human body under test suffers from. Besides, the health parameter may adopt different data to indicate whether the human body under test suffers from a disease and what kind of disease the human body under test suffers from. In the embodiments of the present disclosure, the physical sign parameter acquisition module may be an input module. A user may input the physical sign parameter of the human body under test by means of the input module, so that the physical sign parameter acquisition module acquires the physical sign parameter.

The processing module may determine the fracture index of the human body under test in accordance with the BMD, the balance degree and the physical sign parameter of the human body under test. Optionally, the processing module may determine the fracture index of the human body under test in accordance with the BMD, the balance degree of the human body under test and a fracture index model. The fracture index model may be built and provided in the processing module in advance. The processing module may substitute the BMD, the balance degree and the physical sign parameter of the human body under test into the fracture index model. The fracture index of the human body under test is obtained through calculation.

Furthermore, continuously referring to FIG. 5, the fracture index determination system further comprises a motion item determination module 005 that is connected to the motion balance degree acquisition unit.

The procedure of acquiring the motion balance degree of the human body under test by means of the motion balance degree acquisition unit when the human body under test is in the moving state on the bearing unit may comprise: determining an motion item that corresponds to the human body under test by means of the motion item determination module in accordance with the BMD and the physical sign parameter of the human body under test; and acquiring the motion balance degree of the human body under test by means of the motion balance degree acquisition unit when the human body under test performs the motion item that corresponds to the human body under test on the bearing unit.

For example, the motion item may comprise motion type and motion intensity. The motion type is test type that may comprise the platform motion balance degree test, the slope motion balance degree test and the across-obstacle motion balance degree test. The motion intensity may comprise primary intensity, secondary intensity, tertiary intensity and the like. The primary intensity indicates a relatively lower motion intensity. The secondary intensity indicates an appropriate motion intensity. The tertiary intensity indicates a relatively higher motion intensity.

Optionally, a BMD threshold may be set in the motion item determination module which may determine the motion item that corresponds to the human body under test in accordance with the relationship between the BMD of the human body under test and the BMD threshold. Optionally, when the BMD of the human body under test is more than or equal to the BMD threshold, it is believed that the human body under test has a relatively higher BMD. At this time, the motion item determination module determines the motion item with the relatively higher motion intensity as the motion item that corresponds to the human body under test. When the BMD of the human body under test is less than the BMD threshold, it is believed that the human body under test has a relatively lower BMD. At this time, the motion item determination module determines the motion item with the relatively lower motion intensity as the motion item that corresponds to the human body under test. In some embodiments, a corresponding relationship between the physical sign parameter and the motion item may be stored in the motion item determination module, which may inquire the corresponding relationship between the physical sign parameter and the motion item in accordance with the physical sign parameter of the human body under test to obtain the motion item that corresponds to the human body under test.

Furthermore, continuously referring to FIG. 5, the fracture index determination system further comprises a three-dimensional motion capture module 006 and a presentation module 007 that are connected to the processing module 003 respectively. The fracture index determination method further comprises the following procedures:

capturing a motion action of the human body under test by means of the three-dimensional motion capture module when the human under test performs the motion item that corresponds to the human body under test on the bearing unit; generating test guidance information in accordance with the motion action of the human body under test, wherein the test guidance information is used to guide the human body under test to finish the motion item that corresponds to the human body under test; and presenting the test guidance information by means of the presentation module.

Here, the three-dimensional motion capture module may comprise a plurality of cameras. The cameras may continuously capture the motion action of the human body under test when the human body under test performs the motion item that corresponds to the human body under test on the bearing unit. Action information for indicating the motion action is generated in accordance with the motion action of the human body under test and sent to the processing module The processing module may compare the action information sent by the three-dimensional motion capture module with self-stored action information to determine whether there exists a motion action that is identical with the motion action of the human body under test in the motion action indicated by the self-stored action information. If yes, the processing module determines the motion action of the human body under test as a correct motion action. If not, the processing module determines the motion action of the human body under test as a false motion action. At this time, the processing module may generate the test guidance information that is used to indicate the motion action. The motion action indicated by the test guidance information is the motion action that is nearest to the motion action of the human body under test in the motion action indicated by the action information stored in the processing module. The presentation module may present the test guidance information in the format of text displaying, voice broadcasting or video displaying to guide the human body under test to make a correct motion action.

Furthermore, after the step 603, the fracture index determination method further comprise the following procedures:

determining an exercise plan of the human body under test in accordance with the fracture index of the human body under test; and presenting the exercise plan by means of the presentation module.

Optionally, a corresponding relationship between a fracture index interval and the exercise plan may be stored in the processing module. The processing module may determine the fracture index interval to which the fracture index of the human body under test belongs first, and then inquires the corresponding relationship between the fracture index interval and the exercise plan in accordance with the fracture index interval to which the fracture index of the human body under test belongs to obtain the exercise plan for the human body under test. The presentation module may present the exercise plan after the processing module determines the exercise plan for the human body under test. Optionally, the presentation module may present the exercise plan in the format of text displaying or voice broadcasting.

To sum up, in the fracture index determination method provided by the embodiments of the present, the processing module may acquire the BMD of the human body under test through the BMD acquisition module, acquire the balance degree of the human body under test through the balance degree acquisition module and determine the fracture index of the human body under test in accordance with the BMD and the balance degree of the human body under test. In the embodiments of the present disclosure, as the fracture index is determined through the BMD and the balance degree of the human body under test, the problem of a relatively lower accuracy in determining the fracture index is solved. The accuracy in determining the fracture index is improved.

It should be noted that the fracture index determination method and the fracture index determination system which are provided by the embodiments have the same concept. For details not disclosed in the fracture index determination method, please refer to the embodiments of the fracture index determination system, which will not be repeated herein.

The embodiments of the present disclosure further provide a non-temporary computer readable storage medium, wherein an instruction is stored in the non-temporary computer readable storage medium, and when the instruction in the non-temporary computer readable storage medium is operated on a processing component, the processing component executes the fracture index determination methods in the above embodiments.

The foregoing are only exemplary embodiments of the present disclosure, and are not intended to limit the present disclosure. Within the spirit and principles of the disclosure, any modifications, equivalent substitutions, improvements, etc., are within the scope of protection of the present disclosure.

What is claimed is:

1. A fracture index determination system, comprising:
a bone mineral density (BMD) acquisition module, a balance degree acquisition module and a processing module, wherein
the BMD acquisition module and the balance degree acquisition module are connected to the processing module respectively;
the BMD acquisition module is configured to acquire a BMD of a human body under test;
the balance degree acquisition module is configured to acquire a balance degree of the human body under test, and the balance degree is used to characterize the standing stability of the human body; and
the processing module is configured to determine a fracture index of the human body under test in accordance with the BMD and the balance degree of the human body under test.

2. The fracture index determination system according to claim 1, further comprising:
a physical sign parameter acquisition module connected to the processing module and configured to acquire a physical sign parameter of the human body under test, wherein
the processing module is configured to determine the fracture index of the human body under test in accordance with the BMD, the balance degree and the physical sign parameter of the human body under test.

3. The fracture index determination system according to claim 1, wherein
the balance degree comprises at least one of a static balance degree and a motion balance degree;
the balance degree acquisition module comprises a bearing unit, a static balance degree acquisition unit and a motion balance degree acquisition unit, and the static balance degree acquisition unit and the motion balance degree acquisition unit are connected to the processing module respectively;
the bearing unit is configured to bear the human body under test;
the static balance degree acquisition unit is configured to acquire a static balance degree of the human body under test when the human body under test is in a static state on the bearing unit; and
the motion balance degree acquisition unit is configured to acquire a motion balance degree of the human body under test when the human body under test is in a moving state on the bearing unit.

4. The fracture index determination system according to claim 3, wherein
the static balance degree acquisition unit comprises a pressure sensor and a processing sub-unit, and the pressure sensor is disposed on the bearing unit and connected to the processing sub-unit;
the pressure sensor is configured to acquire pressure applied by the human body under test to the bearing unit when the human body under test is in the static state on the bearing unit; and
the processing sub-unit is configured to determine a variation of the pressure applied by the human body under test to the bearing unit in accordance with the pressure applied by the human body under test to the bearing unit within a test period and to determine the static balance degree of the human body under test in accordance with the variation of the pressure.

5. The fracture index determination system according to claim 3, wherein
the motion balance degree acquisition unit comprises a camera and a processing sub-unit, and the camera is disposed in a target position, a preset distance away from the bearing unit, and is connected to the processing sub-unit;
the camera is configured to acquire a motion image of the human body under test when the human body under test is in the moving state on the bearing unit; and
the processing sub-unit is configured to determine motion information of the human body under test in accordance with the motion image of the human body under test and to determine the motion balance degree of the human body under test in accordance with the motion information of the human body under test.

6. The fracture index determination system according to claim 3, further comprising a motion item determination module connected to the motion balance degree acquisition unit;
the motion item determination module is configured to determine a motion item that corresponds to the human body under test in accordance with the BMD or the physical sign parameter of the human body under test; and
the motion balance degree acquisition unit is configured to acquire the motion balance degree of the human body under test when the human body under test performs the motion item that corresponds to the human body under test on the bearing unit.

7. The fracture index determination system according to claim 6, further comprising a three-dimensional motion capture module and a presentation module that are connected to the processing module respectively;
the three-dimensional motion capture module is configured to capture a motion action of the human body under test when the human body under test performs the motion item that corresponds to the human body under test on the bearing unit;
the processing module is configured to generate test guidance information in accordance with the motion action of the human body under test, and the test guidance information is used to guide the human body under test to finish the motion item that corresponds to the human body under test; and
the presentation module is configured to present the test guidance information.

8. The fracture index determination system according to claim 7, wherein
the processing module is further configured to determine an exercise plan for the human body under test in accordance with the fracture index of the human body under test; and
the presentation module is further configured to present the exercise plan.

9. The fracture index determination system according to claim 1, wherein
the processing module is configured to determine the fracture index of the human body under test in accordance with the BMD, the balance degree of the human body under test and a fracture index model.

10. The fracture index determination system according to claim 8, further comprising a communication module connected to the processing module, wherein
the communication module is configured to upload the fracture index of the human body under test and the exercise plan for the human body under test to a server.

11. A fracture index determination method, comprising:
acquiring a bone mineral density (BMD) of a human body under test;
acquiring a balance degree of the human body under test, wherein the balance degree is used to characterize the standing stability of the human body; and
determining a fracture index of the human body under test in accordance with the BMD and the balance degree of the human body under test.

12. The fracture index determination method according to claim 11, further comprising:
acquiring a physical sign parameter of the human body under test, wherein
said determining the fracture index of the human body under test in accordance with the BMD and the balance degree of the human body under test comprises:
determining the fracture index of the human body under test in accordance with the BMD, the balance degree and the physical sign parameter of the human body under test.

13. The fracture index determination method according to claim 11, wherein said acquiring the balance degree of the human body under test comprises:
acquiring a static balance degree of the human body under test when the human body under test is in a static state on a bearing unit; and
acquiring a motion balance degree of the human body under test when the human body under test is in a moving state on the bearing unit.

14. The fracture index determination method according to claim 13, wherein
said acquiring the static balance degree of the human body under test when the human body under test is in the static state on the bearing unit comprises:
acquiring pressure applied by the human body under test to the bearing unit when the human body under test is in the static state on the bearing unit; and
determining a variation of the pressure applied by the human body under test to the bearing unit in accordance with the pressure applied by the human body under test to the bearing unit within a test period and determining the static balance degree of the human body under test in accordance with the variation of the pressure.

15. The fracture index determination method according to claim 13, wherein
said acquiring the motion balance degree of the human body under test when the human body under test is in the moving state on the bearing unit comprises:
acquiring a motion image of the human body under test when the human body under test is in the moving state on the bearing unit; and
determining motion information of the human body under test in accordance with the motion image of the human body under test and determining the motion balance degree of the human body under test in accordance with the motion information of the human body under test.

16. The fracture index determination method according to claim 13, further comprising:
determining a motion item that corresponds to the human body under test in accordance with the BMD or the physical sign parameter of the human body under test, wherein
said acquiring the motion balance degree of the human body under test when the human body under test is in the moving state on the bearing unit comprises:
acquiring the motion balance degree of the human body under test when the human body under test performs the motion item that corresponds to the human body under test on the bearing unit.

17. The fracture index determination method according to claim 16, further comprising:
capturing a motion action of the human body under test when the human body under test performs the motion item that corresponds to the human body under test on the bearing unit;
generating test guidance information in accordance with the motion action of the human body under test, wherein the test guidance information is used to guide the human body under test to perform the motion item that corresponds to the human body under test; and
presenting the test guidance information.

18. The fracture index determination method according to claim 17, further comprising:
determining an exercise plan for the human body under test in accordance with the fracture index of the human body under test; and
presenting the exercise plan.

19. The fracture index determination method according to claim 11, wherein said determining the fracture index of the human body under test in accordance with the BMD and the balance degree of the human body under test comprises:
determining the fracture index of the human body under test by means of a processing module in accordance with the BMD, the balance degree of the human body under test and a fracture index model.

20. A non-temporary computer readable storage medium, wherein an instruction is stored in the non-temporary computer readable storage medium, and when the instruction in the non-temporary computer readable storage medium is operated on a processing component, the processing component executes the following steps:
acquiring a bone mineral density (BMD) of a human body under test;
acquiring a balance degree of the human body under test, wherein the balance degree is used to characterize the standing stability of the human body; and
determining a fracture index of the human body under test in accordance with the BMD and the balance degree of the human body under test.
